(19) United States
(12) Patent Application Publication
(10) Pub. No.: US 2023/0000615 A1
(43) Pub. Date: Jan. 5, 2023

(54) ARTHROSCOPIC ACL REPAIR SYSTEM AND METHOD

(71) Applicant: Miach Orthopaedics, Inc., Westborough, MA (US)

(72) Inventors: Martha SHADAN, Westborough, MA (US); Rita PAPARAZZO, Hopkinton, MA (US); Stephen WOHLERT, Westborough, MA (US)

(73) Assignee: Miach Orthopaedics, Inc., Westborough, MA (US)

(21) Appl. No.: 17/854,098

(22) Filed: Jun. 30, 2022

Related U.S. Application Data
(60) Provisional application No. 63/217,208, filed on Jun. 30, 2021.

(51) Int. Cl. A61F 2/08; A61B 17/04; A61L 27/24

(52) U.S. Cl. CPC A61F 2/0811; A61B 17/0401; A61L 27/24; A61L 2430/10

(57) ABSTRACT

An arthroscopic system for the repair of a ruptured anterior cruciate is provided. Aspects of the invention include a scaffold attached by a suture to an fixation device and inserted into a repair site via arthroscopic equipment. The scaffold and suture are further secured near or at the repair site via arthroscopic equipment having an elongated delivery member that contains the scaffold and suture prior to inserting the scaffold and suture into the repair site.

### CROSS-REFERENCE TO RELATED APPLICATIONS

The present application claims priority to and the benefit of U.S. Provisional Patent Application No. 63/217,208, filed Jun. 30, 2021, the contents of which are hereby incorporated in their entirety.

### FIELD OF THE INVENTION

The invention relates generally to systems and methods for the repair of a ruptured ligament utilizing an arthroscopic repair system.

### BACKGROUND OF THE INVENTION

Intra-articular tissues, such as the anterior cruciate ligament (ACL), do not heal after rupture. In addition, the meniscus and the articular cartilage in human joints also often fail to heal after an injury. Tissues found outside of joints heal by forming a fibrin clot, which connects the ruptured tissue ends and is subsequently remodeled to form scar, which heals the tissue. Inside a synovial joint, a fibrin clot either fails to form or is quickly lysed after injury to the knee, thus preventing joint arthrosis and stiffness after minor injury. Joints contain synovial fluid which, as part of normal joint activity, naturally prevent clot formation in joints. This fibrinolytic process results in premature loss of the fibrin clot scaffold and disruption of the healing process for tissues within the joint or within intra-articular tissues.

The current treatment method for human anterior cruciate ligament repair after rupture involves removing the ruptured fan-shaped ligament and replacing it with a point-to-point tendon graft (ACL reconstruction). While this procedure can initially restore gross stability in most patients, longer follow-up demonstrates many post-operative patients have abnormal structural laxity, suggesting the reconstruction may not withstand the physiologic forces applied over time (Dye, 325 Clin. Orthop. 130-139 (1996)). The loss of anterior cruciate ligament function has been found to result in early and progressive radiographic changes consistent with joint deterioration (Hefti et al., 73A(3) J. Bone Joint Surg. 373-383 (1991)), and over 70% of patients undergoing ACL reconstruction develop osteoarthritis at only 14 years after injury (von Porat et al., Ann Rheum Dis. 63(3):269-73 (2004)). As anterior cruciate ligament rupture is most commonly an injury of a young athletes in their teens and twenties, early osteoarthritis in this group has difficult consequences.

Current ACL repair methods and treatments may include systems and devices utilized in surgery.

### SUMMARY OF THE INVENTION

There is a need to provide an efficient delivery mechanism of ACL repair devices utilizing arthroscopic repair systems. The invention relates, in some aspects, to methods and products that facilitate anterior cruciate ligament regeneration or healing using an arthroscopic repair system.

An embodiment of the present disclosure includes an arthroscopic repair system. The arthroscopic repair system includes a tissue healing device configured to repair a ligament at a repair site. The tissue healing device includes an implantable material configured to be positioned between a ruptured end of the ligament and a bone. The tissue healing device further includes at least one fixation device configured to be secured to the bone. The tissue healing device further includes at least one suture configured to be threaded through or along the implantable material to position the implantable material between to the ruptured end of the ligament and the bone. The at least one suture is attached to the at least one fixation device. The arthroscopic repair system includes arthroscopic equipment sized and shaped to contain the at least one suture and the implantable material. The arthroscopic equipment is configured to a) insert the suture through the implantable material, and b) position the implantable material between the ruptured end of the ligament and the bone.

A further embodiment of the present disclosure includes a system for repair of an anterior cruciate ligament. The system includes a fixation device capable of forming a stable attachment to a first bone at a repair site. The system further includes a suture having a first end and a second end. The second end is attachable to a ruptured end of the ligament at the repair site. The ligament is configured to be connected to a second bone. The system further includes a scaffold, wherein the scaffold consists essentially of a porous sponge scaffold, wherein the scaffold is threaded onto the suture. The system further includes an elongated delivery member having a channel that extends from a proximal end to a distal end. The suture and the scaffold are contained within the channel such that the scaffold is positionable along the suture.

Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE DRAWINGS

The figures are illustrative only and are not required for enablement of the invention disclosed herein.

FIG. 1A is a diagrammatic representation of a torn anterior cruciate ligament;

FIG. 1B is a diagrammatic representation of a scaffold device having an fixation device and attached sutures;

FIG. 1C is a diagrammatic representation of a scaffold device implanted into a repair site around a ruptured ACL;

FIG. 2A is a diagrammatic representation of a suture fixation device inserted into a femur;

2B is a diagrammatic representation of a drill hole in a femur and sutures attached to the opposite surface of the femur;

FIG. 2C is a diagrammatic representation of a staple affixing a suture into a notch;

FIG. 2D is a diagrammatic representation of an fixation device with a central hole to allow bone marrow bleeding to flow into the attached scaffold;

FIG. 2E is a diagrammatic representation of an fixation device with a scaffold sponge swaged directly onto it;

FIG. 3A is a diagrammatic representation of a suture attached through a drill hole in a bone;

FIG. 3B is a diagrammatic representation of an fixation device inserted into a bone;

FIG. 4 is a diagrammatic representation of the arthroscopic repair system according to an embodiment of the present disclosure;

FIG. 5A is a schematic showing the ruptured ligament shown in FIGS. 1A-3B;

FIG. 5B is a schematic showing the repair device inserted into the repair site using the arthroscopic repair system shown in FIG. 4; and

FIG. 5C is a schematic showing the sutures, the fixation device, and the scaffold of the repair device shown in FIGS. 1A-3B being secured into the repair site.

### DETAILED DESCRIPTION OF THE INVENTION

Aspects of the invention relate to systems and methods for repairing a ruptured anterior cruciate ligament (“ACL”). The system includes a scaffold configured for the repair of the ruptured ligament, an fixation device, and includes a suture. The scaffold allows the subject's body to develop a network of capillaries, arteries, and veins. Well-vascularized connective tissues heal as a result of migration of fibroblasts into the scaffold. The methods and systems of the present disclosure provides a connection between the ruptured ligament, or forms around the torn ligament, and promotes the repair of the ruptured or torn ligament while maintaining the integrity and structure of the ligament.

The present disclosure provides a three-dimensional (3-D) scaffold for repairing a ruptured or torn ACL. The scaffold provides a connection between the ruptured ends of the ligament and fibers, or forms around the torn ligament, after injury, and encourages the migration of appropriate healing cells to form scar and new tissue in the scaffold. The scaffold is a bioengineered substitute for a fibrin clot and is implanted, for example, between the ruptured ends of the ligament fascicles, or placed around the torn ligament. This substitute scaffold is designed to stimulate cell proliferation and extracellular matrix production in the gap between the ruptured ends of the ligament or the tear in the ligament, thus facilitating healing and regeneration.

As used herein, the injury may be a torn ligament or a ruptured ligament. A torn ligament may be a partial tear. A torn ligament may also refer to a complete tear. A partial tear is one where a portion of the ligament is damaged, but the ligament remains connected. The tear may be of any length or shape. A ruptured ligament, also known as a complete tear, is one where the ligament has been completely severed providing two separate ends of the ligament. A ruptured ligament may provide two ligament ends of similar or different lengths. The rupture may be such that a ligament stump is formed at one end. For example, there may be a tibial stump connected to the tibia and a femoral stump connected to the femur.

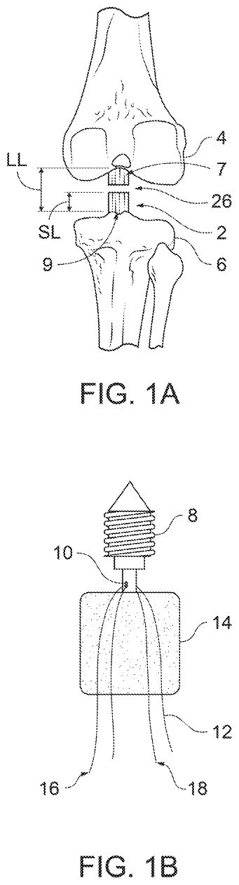

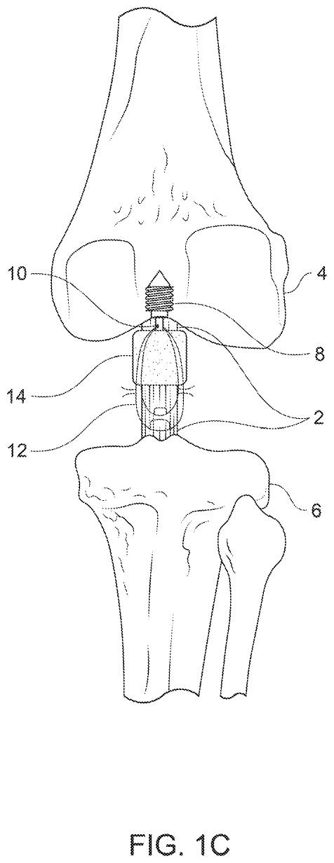

An example of a ruptured anterior cruciate ligament is depicted in FIG. 1A. The anterior cruciate ligament (ACL) 2 is one of four strong ligaments that connects the bones of the knee joint. The function of the ACL is to provide stability to the knee and minimize stress across the knee joint. It restrains excessive forward movement of the lower leg bone, the tibia 6, in relation to the thigh bone, the femur 4, and limits the rotational movements of the knee.

As shown in FIGS. 1A-3B, the anterior cruciate ligament 2 is ruptured such that it no longer forms a connection between the femur bone 4 and the tibia bone 6. The resulting ends of the ruptured ACL 2 may be of any length. The ends may be of a similar length, or one end may be longer in length than the other. The end on the femur 4 includes the femoral ACL stump 7. The end on the tibia 6 includes a tibial stump 9. In some instances, it is believed that a repair is desirable when the tibial stump length SL is less than about 75% of the effective ligament length LL but greater than 5% of a total length LL of the ACL. The total length of the ACL is considered to be the length of ligament from femoral footprint to the tibial footprint along a linear axis.

The knee joint includes tibial spines on the tibia 6 and the intercondylar notch of the femur 4. In some instances, the methods as described herein may include performing a notchplasty of the intercondylar notch of the femur to provide space for larger ligament to form after surgical repair using a scaffold. Such a notchplasty improves the size of the healing ligament, specifically resulting in a larger cross-sectional area of the ligament. As the mechanical strength of a ligament, and subsequently its ability to maintain the distance between the femur and tibia, is directly correlated with its cross sectional area, enlarging the notch with a notchplasty can help make a stronger repaired ACL and has been found by the inventors to be beneficial in ACL repair using a scaffold as described in the present disclosure.

A scaffold of the present disclosure can be any shape that is useful for implantation into a subject. The scaffold, for instance, can be tubular, semi-tubular, cylindrical, including either a solid cylinder or a cylinder having hollow cavities, a tube, a flat sheet rolled into a tube so as to define a hollow cavity, liquid, an amorphous shape which conforms to that of the repair space, a “Chinese finger trap” design, a trough shape, or square. Other shapes suitable for the scaffold of the device as known to those of ordinary skill in the art are also contemplated in the invention.

The present disclosure includes a scaffold 14, such that the scaffold 14 is configured for repair. The scaffold 14 is capable of being inserted into an area requiring repair and promotes regeneration of the ligament. The scaffold 14 is capable of insertion into a repair site and either forming a connection between the ends of the ruptured ligament, between bone, or forming around the torn ligament such that the integrity and structure of the ligament is maintained. Regeneration offers several advantages over reconstruction, previously used in ligament repair, including maintenance of the complex insertion sites and fan-shape of the ligament, and preservation of remaining proprioceptive fibers within the ligament substance.

Referring to FIGS. 1A-3E, the arthroscopic repair system of the present disclosure includes a repair device 1 and arthroscopic equipment 30. An example of a repair device 1 is depicted in FIGS. 1B and 1C. For example, the scaffold 14 is attached to a suture 12 and a fixation device 8. The fixation device 8 may, as shown in FIGS. 1B and 1C, be attached to the suture 12 through an eyelet 10 of the fixation device 8. In this configuration, the fixation device 8 is attached into a bone. The bone may be either the femur 4 or the tibia 6.

The scaffold 14 may function either as an insoluble or biodegradable regulator of cell function or simply as a delivery vehicle of a supporting structure for cell migration or synthesis. Numerous matrices made of either natural or synthetic components have been investigated for use in ligament repair and reconstruction. Natural matrices are made from processed or reconstituted tissue components (such as collagens and GAGs). Because natural matrices mimic the structures ordinarily responsible for the reciprocal interaction between cells and their environment, they act as cell regulators with minimal modification, giving the cells the ability to remodel an implanted material, which is a prerequisite for regeneration.

Synthetic matrices are made predominantly of polymeric materials. Synthetic matrices offer the advantage of a range of carefully defined chemical compositions and structural arrangements. Some synthetic matrices are not degradable. While the non-degradable matrices may aid in repair, non-degradable matrices are not replaced by remodeling and therefore cannot be used to fully regenerate ligament. It is also undesirable to leave foreign materials permanently in a joint due to the problems associated with the generation of wear particles, thus degradable materials are preferred for work in regeneration. Degradable synthetic scaffolds can be engineered to control the rate of degradation.

The scaffold 14 is preferably made of a compressible, resilient material which has some resistance to degradation by synovial fluid. Synovial fluid as part of normal joint activity, naturally prevents clot formation. This fibrinolytic process would result in the premature degradation of the scaffold and disrupt the healing process of the ligament. The material may be either permanent or biodegradable material, such as polymers and copolymers. The scaffold 14 can be composed, for example, of collagen fibers, collagen gel, foamed rubber, natural material, synthetic materials such as rubber, silicone and plastic, ground and compacted material, perforated material, or a compressible solid material.

The scaffold 14 may be a solid material such that its shape is maintained, or a semi-solid material capable of altering its shape and or size. The scaffold 14 may be made of expandable material allowing it to contract or expand as required. The material can be capable of absorbing plasma, blood, other body fluids, liquid, hydrogel, or other material the scaffold either comes into contact with or is added to the scaffold.

The scaffold material can be protein, lyophilized material, or any other suitable material. A protein can be synthetic, bioabsorbable or a naturally occurring protein. A protein includes, but is not limited to, fibrin, hyaluronic acid, elastin, extracellular matrix proteins, or collagen. The scaffold material may be plastic or self-assembling peptides. The scaffold material may incorporate therapeutic proteins including, but not limited to, hormones, cytokines, growth factors, clotting factors, anti-protease proteins (e.g., alpha1-antitrypsin), angiogenic proteins (e.g., vascular endothelial growth factor, fibroblast growth factors), antiangiogenic proteins (e.g., endostatin, angiostatin), and other proteins that are present in the blood, bone morphogenic proteins (BMPs), osteoinductive factor (IFO), fibronectin (FN), endothelial cell growth factor (ECGF), cementum attachment extracts (CAE), ketanserin, human growth hormone (HGH), animal growth hormones, epidermal growth factor (EGF), interleukin-1 (IL-1), human alpha thrombin, transforming growth factor (TGF-beta), insulin-like growth factor (IGF-1), platelet derived growth factors (PDGF), fibroblast growth factors (FGF, bFGF, etc.), and periodontal ligament chemotactic factor (PDLGF), for therapeutic purposes. A lyophilized material is one that is capable of swelling when liquid, gel or other fluid is added or comes into contact with it.

Many biological materials are available for making the scaffold, including collagen compositions (either collagen fiber or collagen gel), compositions containing glycosaminoglycan (GAG), hyaluronan compositions, and various synthetic compositions. Collagen-glycosaminoglycan (CG) copolymers have been used successfully in the regeneration of dermis and peripheral nerve. Porous natural polymers, fabricated as sponge-like and fibrous scaffolds, have been investigated as implants to facilitate regeneration of selected musculoskeletal tissues including ligaments. In one embodiment, the scaffold 14 is a sponge scaffold made from tendon (xenograft, allograft, autograft) or ligament or skin or other connective tissue which could be in the native state or processed to facilitate cell ingrowth or other biologic features.

In the illustrated embodiment the scaffold 14 is composed of a sponge or sponge-like material. The sponge scaffold 14 may be absorbable or nonabsorbable. The sponge scaffold 14 may include collagen, elastin, extracellular matrix protein, plastic, or self-assembling peptides. The sponge scaffold 14 may be hydrophillic. The sponge scaffold 14 is capable of compression and expansion as desired. For example, the sponge scaffold 14 may be compressed prior to or during implantation into a repair site. A compressed sponge scaffold allows for the sponge scaffold to expand within the repair site. The sponge may be lyophilized and/or compressed when placed in the repair site and expanded once in place. The expansion of the sponge scaffold 14 may occur after contact with blood or other fluid in the repair site or added to the repair site.

The sponge scaffold 14 may also be porous. The sponge scaffold 14 may be saturated or coated with a liquid, gel, or hydrogel repair material prior to implantation into a repair site. Coating or saturation of a sponge scaffold may ease implantation into a relatively undefined defect area as well as help to fill a particularly large defect area. The sponge scaffold 14 may be composed of collagen. In a preferred embodiment, the sponge scaffold 14 is treated with hydrogel. Examples of scaffolds and repair materials useful according to the invention are found in U.S. Pat. No. 6,964,685 and U.S. Patent Application Nos. 2004/0059416 and 2005/0261736, the entire contents of each are herein incorporated by reference.

An important subset of natural matrices are those made predominantly from collagen, the main structural component in ligament. Collagen can be of the soluble or the insoluble type. Preferably, the collagen is soluble, e.g., acidic or basic. For example, the collagen can be type I, II, III, IV, V, IX or X. Preferably the collagen is type I. More preferably the collagen is soluble type I collagen. Type I collagen is the predominant component of the extracellular matrix for the human ACL and provides an example of a choice for the basis of a bioengineered scaffold. Collagen occurs predominantly in a fibrous form, allowing design of materials with very different mechanical properties by altering the volume fraction, fiber orientation, and degree of cross-linking of the collagen. The biologic properties of cell infiltration rate and scaffold degradation may also be altered by varying the pore size, degree of cross-linking, and the use of additional proteins, such as glycosaminoglycans, growth factors, and cytokines. In addition, collagen-based biomaterials can be manufactured from a patient's own skin, thus minimizing the antigenicity of the implant (Ford et al., 105 Laryngoscope 944-948 (1995)).

The present disclosure may also include one or more fixation devices 8. The fixation device 8 is a device capable of insertion into the bone such that it forms a stable attachment to the bone. In some instances, the fixation device 8 is capable of being removed from the bone if desired. The fixation device 8 may be conical shaped having a sharpened tip at one end and a body having a longitudinal axis. The body of the fixation device 8 may increase in diameter along its longitudinal axis. The body of the fixation device 8 may include grooves suitable for screwing the fixation device 8 into position. For example, as depicted in FIG. 1C, the fixation device 8 is screwed into the femur bone 4. The fixation device 8 may include an eyelet 10 at the base of the fixation device body through which one or more sutures may be passed. The eyelet 10 may be oval or round and may be of any size suitable to allow one or more sutures to pass through and be held within the eyelet 10.

The fixation device 8 may be attached to a bone by physical or mechanical methods as known to those of ordinary skill in the art. The fixation device 8 includes, but is not limited to, a screw, a barb, an anchor, a helical anchor, a staple, a clip, a snap, a rivet, an endobutton, or a crimp-type anchor. The body of the fixation device 8 may be varied in length. Examples of fixation devices, include but are not limited to, IN-FAST™ Bone Screw System (Influence, Inc., San Francisco, Calif.), IN-TAC™ Bone Anchor System (Influence, Inc., San Francisco, Calif.), Model 3000 AXYALOOP™ Titanium Bone Anchor (Axya Medical Inc., Beverly, Mass.), OPUS MAGNUM® Anchor with Inserter (Opus Medical, Inc., San Juan Capistrano, Calif.), ANCHRON™, HEXALON™, TRINION™ (all available from Inion Inc., Oklahoma City, Okla.) and TwinFix AB absorbable suture anchor (Smith & Nephew, Inc., Andover, Mass.). Fixation devices are available commercially from manufacturers such as Influence, Inc., San Francisco, Calif., Axya Medical Inc., Beverly, Mass., Opus Medical, Inc., San Juan Capistrano, Calif., Inion Inc., Oklahoma City, Okla., and Smith & Nephew, Inc., Andover, Mass.

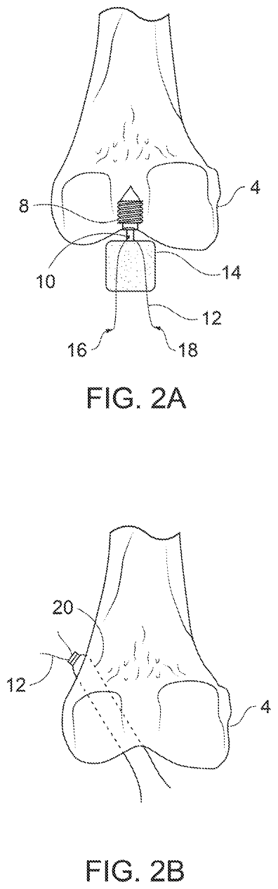

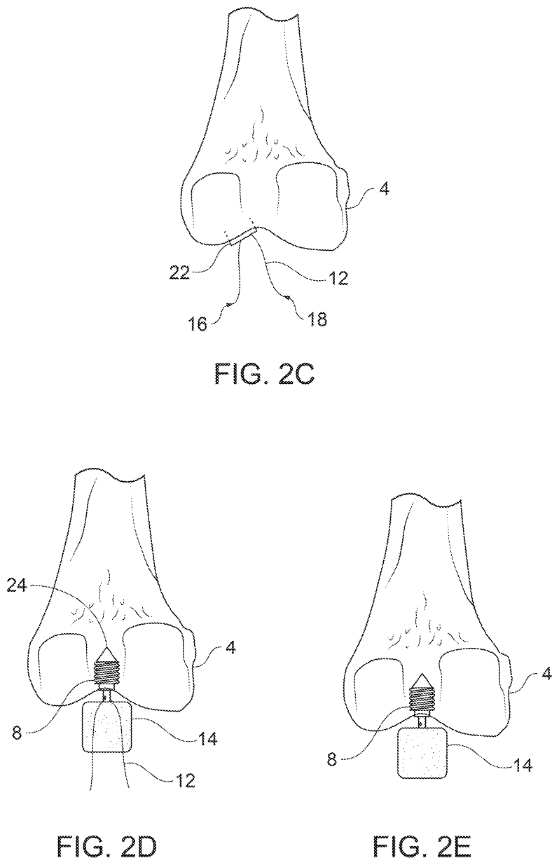

The fixation device 8 may be attached directly to the scaffold 14 where the fixation device 8 is swaged directly onto the scaffold 14. FIG. 2E depicts such an example. The fixation device 8 is attached directly to the scaffold 14 by its base end and the fixation device 8 is attached to the femur 4 by its sharpened end.

The fixation device 8 may be attached indirectly to the scaffold 14 using the suture 12 to secure it in position. FIG. 2A depicts such an example. The suture 12 is passed through the eyelet 10 of the fixation device 8 and held within the eyelet 10 to attach the scaffold 14. The first end 16 and the second end 18 of the suture 12 are free and emerge from the scaffold 14. The fixation device 8 is attached to the femur 4 by its sharpened end.

The fixation device 8 may be composed of a non-degradable material, such as metal, for example titanium 316 LVM stainless steel, CoCrMo alloy, or Nitinol alloy, or plastic. The fixation device 8 is preferably bioabsorbable such that the subject is capable of breaking down the fixation device 8 and absorbing it. Examples of bioabsorbable material include, but are not limited to, MONOCRYL (poliglecaprone 25), PDS II (polydioxanone), surgical gut suture (SGS), gut, coated VICRYL (polyglactin 910, polyglactin 910 braided), human autograft tendon material, collagen fiber, POLYSORB, poly-L-lactic acid (PLLA), polylactic acid (PLA), polysulfone, polylactides (Pla), racemic form of polylactide (D,L-Pla), poly(L-lactide-co-D,L-lactide), 70/30 poly(L-lactide-co-D,L-lactide), polyglycolides (PGa), polyglycolic acid (PGA), polycaprolactone (PCL), polydioxanone (PDS), polyhydroxyacids, and resorbable plate material (see e.g. Orthopedics, October 2002, Vol. 25, No. 10/Supp.). The fixation device 8 may be bioabsorbed over a period of time which includes, but is not limited to, days, weeks, months or years.

The fixation device 8 may have a central hole 24 through which fluids, such as blood, may pass. The hole 24 may allow such fluids to flow onto the attached scaffold 14. FIG. 2D depicts such an example. The fixation device 8 is attached to the femur 4 and includes a central hole 24 through which fluids, such as blood, may pass. Blood is able to pass through the central hole 24 in the fixation device 8 and onto the scaffold 14 which absorbs the blood.

In the illustrated embodiment, the fixation device 8 is attached to the scaffold 14 using the suture 12. FIG. 1B illustrates an example of the fixation device 8 attached to the scaffold 14 using the suture 12. The suture 12 is passed through the eyelet 10 of an fixation device 8 such that the fixation device 8 is attached to the scaffold 14 by the suture 12. The suture 12 has at least one free end. In some embodiments, a suture has two free ends, a first end 16 and a second end 18.

In one embodiment, the suture 12 is bioabsorbable, such that the subject is capable of breaking down the suture and absorbing it, and synthetic such that the suture may not be from a natural source. In other embodiments, the suture 12 may be permanent such that the subject is not capable of breaking down the suture and the suture remains in the subject. The suture 12 may be rigid or stiff, or may be stretchy or flexible. The suture 12 may be round in shape and may have a flat cross section. Examples of sutures include, but are not limited to, VICRYL™ polyglactin 910, PANACRYL™ absorbable suture, ETHIBOND® EXCEL polyester suture, PDS® polydioxanone suture and PROLENE® polypropylene suture. Sutures are available commercially from manufacturers such as MITEK PRODUCTS division of ETHICON, INC. of Westwood, Mass.

In the illustrated embodiment, the suture 12 may be attached to one or both ends of a ruptured ligament 2 by its first end 16 and/or its second end 18. FIG. 1C illustrates an example of a repair device inserted into a repair site of a ruptured ligament 2. The suture 12 is passed through the eyelet 10 of the fixation device 8 and the first end 16 and second end 18 are tied to the ends of the distal ACL 2. The fixation device 8 is attached to the femur 4 by its sharpened end. The scaffold 14 may be attached to the fixation device 8 by the suture 12 and held in position in the repair site 26. The fixation device 8 may be attached to either the tibia bone 6 or the femur bone 4 to secure the scaffold 14 in position. In alternative embodiments, the scaffold 14 may be attached to the femur bone 4 directly.

A staple 22 is a type of fixation device having two arms that are capable of insertion into a bone. In some instances, the arms of the staple 22 fold in on themselves when attached to the femur 4 or in some instances when attached to other tissue. The staple 22 may be composed of metal, for example titanium or stainless steel, plastic, or any biodegradable material. The staple 22 includes but is not limited to linear staples, circular staples, curved staples or straight staples. Staples are available commercially from manufacturers such as Johnson & Johnson Health Care Systems, Inc. Piscataway, N.J., and Ethicon, Inc., Somerville, N.J. The staple 22 may be attached using any staple device known to those of ordinary skill in the art, for example, a hammer and staple setter (staple holder).

In some embodiments, the staple 22 may be used to hold the suture 12 securely in position. The suture 12 may be attached to the femur 4 using the staple 22 as depicted in FIG. 2C. The suture 12 is held in place in the femur 4 with the staple 22 such that the first end 16 and the second end 18 of the suture 12 are free.

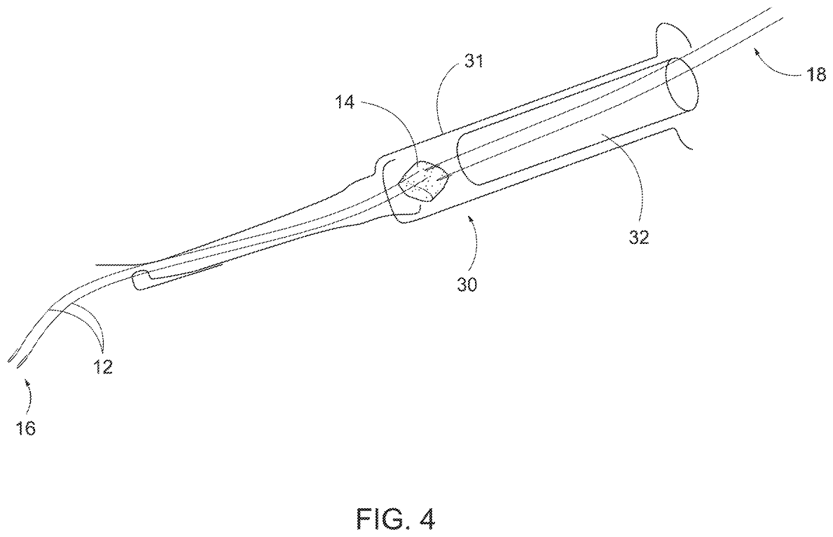

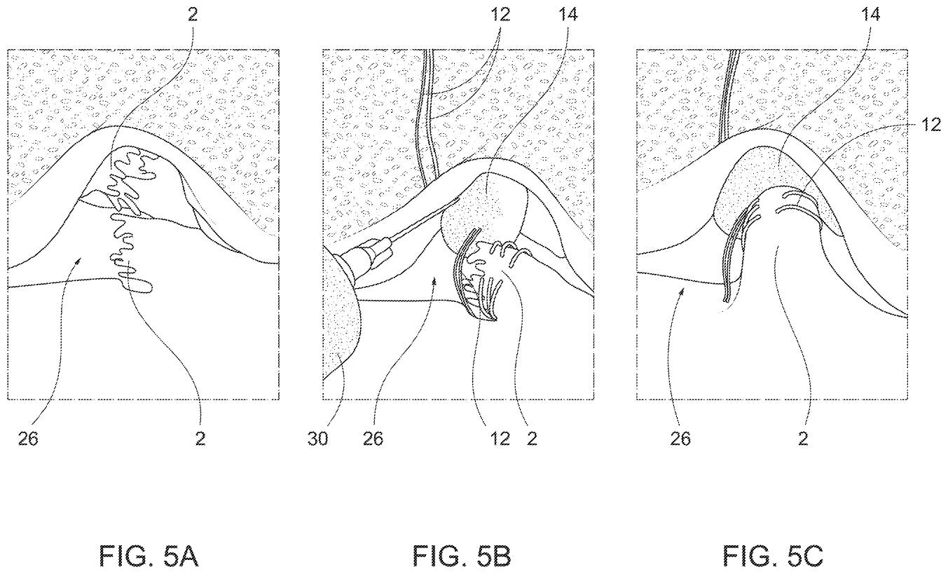

Referring to FIG. 4, the arthroscopic equipment 30 is configured to insert the suture 12 through the scaffold 14. The arthroscopic equipment 30 is further configured to position the scaffold 14 between the ruptured end of the ligament 2 and the bone. The arthroscopic equipment 30 includes a elongated delivery member 31. The elongated delivery member 31 includes a channel that extends from a proximal end to a distal end of the elongated delivery member 31. The elongated delivery member 31 is sized and shaped to contain the scaffold 14 attached to the suture 12 in the channel. At least a portion of the elongated delivery member 31 is further sized and shaped to be capable of being inserted into a repair site.

In the illustrated embodiment, the arthroscopic equipment 30 is a syringe. The syringe may hold the suture 12 and the scaffold 14 in place within the elongated delivery member 31 of the syringe. The syringe may include a plunger 32 configured to push the suture 12, and the scaffold 14 into a repair site such that the scaffold 14 is positioned along the suture 12 between the ruptured end of the ligament 2 and/or the bone. In alternative embodiments, the arthroscopic equipment 30 may include a cannula, a container, and a pressure pump. In another embodiment, the arthroscopic equipment 30 may further include a guiding suture that extends out of the distal end of the elongated delivery member, the guiding suture configured to pull and position the suture and the scaffold into the repair site

Referring to FIGS. 1-4, aspects of the invention relate to methods of repairing a ruptured or torn ligament. In some embodiments, the scaffold 14 and the suture 12 is inserted into a repair site of the ruptured or torn ligament 2 via the arthroscopic equipment 30. In certain embodiments, a hole is drilled into a bone at or near a repair site of the ruptured or torn ligament 2 and the suture 12 is attached through the hole to the bone.

A repair site 26 is the area around a ruptured or torn ligament 2 into which a device may be inserted. The scaffold 14 may be inserted into the repair site 26 during surgery via the arthroscopic equipment 30 using techniques known to those of ordinary skill in the art. The scaffold 14 can either fill the repair site 26 or partially fill the repair site 26. The scaffold 14 can partially fill the repair site 26 when inserted and expand to fill the repair site 26 in the presence of blood, plasma or other fluids either present within or added into the repair site 26.

In one embodiment, the scaffold 14 may be attached directly or indirectly to the femur 4 and may contact the ruptured ligament 2. In another embodiment, the scaffold 14 may form around the ruptured or torn ligament 2 at the repair site 26. For example, in one embodiment, the scaffold 14 may be formed into a tube shape and wrapped around the ligament 2, in another embodiment, the scaffold 14 may be positioned behind the ligament such that the ligament is held within the scaffold 14. In yet another embodiment, the scaffold 14 may be a “Chinese finger trap” design where one end is placed over a stump of a ruptured ligament and the second end placed over the other end of the ruptured ligament.

Aspects of the invention provide methods of repairing the ruptured ligament 2 involving drilling a hole 20 at or near the repair site 26 of the ruptured ligament 2. A bone at or near a repair site is one that is within close proximity to the repair site and can be utilized using the methods and devices of the invention. For example, a bone at or near a repair site of a torn anterior cruciate ligament is a femur 4 bone and/or a tibia 6 bone. The hole 20 can be drilled into a bone using a device such as a Kirschner wire (for example a small Kirschner wire) and drill, or microfracture pics or awls. One or more holes may be drilled into a bone surrounding the repair site 26 to promote bleeding into the repair site 26. The repair can be supplemented by drilling holes into the surrounding bone to cause bleeding. Encouraging bleeding into the repair site may promote the formation of blood clots and enhance the healing process of the injury.

The hole 20 may be drilled into the femur 4 on the opposite side to the repair site 26. The scaffold 14 and the suture 12 may be inserted into the repair site 26 via the arthroscopic equipment 30. The suture 12 may be passed through the hole 20 in the bone and attached to the bone. The ruptured ligament 2 provides two ends of the ligament that were previously connected. In one embodiment, the scaffold 14 may be attached to one or both ends 16, 18 of the ruptured ligament 2 by the suture 12. In another embodiment, the scaffold 14 may be attached to one or both ends of the femur 4 and the tibia 6. The suture 12 may be attached to a second bone site at or near the repair site 26.

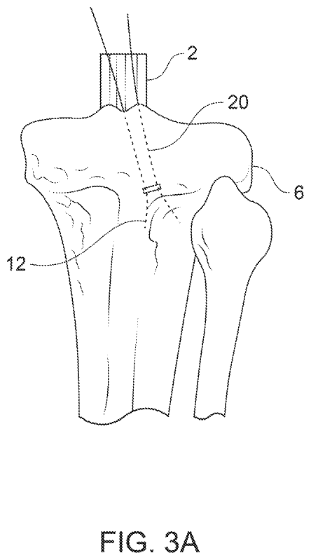

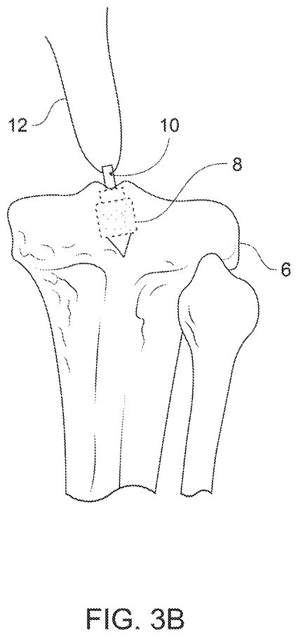

An example of such a method is depicted in FIG. 2B. The hole 20 is drilled into the opposite side of the femur bone 4. The suture 12 is attached to the opposite side of the femur bone 4 using the first end 16 and the second end 18 through the hole 20 via the arthroscopic equipment 30 (not depicted). Another example is depicted in FIG. 3A. A hole 20 is drilled into the tibia 6 near the end of the ruptured ligament 2 and the suture 12 is attached to the tibia 6 through the hole 20 via the arthroscopic equipment 30 (not depicted).

The scaffold 14 can be pretreated with a repair material prior to implantation into a subject. The scaffold 14 may be soaked in a repair material prior to or during implantation into the repair site 26. The repair material may be injected directly into the scaffold 14 prior to or during implantation. The repair material may be injected within a tubular scaffold at the time of repair. Repair material includes, but is not limited to, a gel, for example a hydrogel, a liquid, or collagen. A liquid includes any material capable of forming an aqueous material, a suspension or a solution. The repair material may include additional materials, such as growth factors, antibiotics, insoluble or soluble collagen (in fibrous, gel, sponge or bead form), a cross-linking agent, thrombin, stem cells, a genetically altered fibroblast, platelets, water, plasma, extracellular proteins and a cell media supplement. The additional repair materials may be added to affect cell proliferation, extracellular matrix production, consistency, inhibition of disease or infection, tonicity, cell nutrients until nutritional pathways are formed, and pH of the repair material. All or a portion of these additional materials may be mixed with the repair material before or during implantation, or alternatively, the additional materials may be implanted proximate to the defect area after the repair material is in place.

In certain embodiments, the repair material may include collagen and platelets. In some embodiments, platelets are derived from the subject to be treated. In other embodiments, platelets are derived from a donor that is allogeneic to the subject. In certain embodiments, platelets may be obtained as platelet rich plasma (PRP). In a non-limiting example, platelets may be isolated from a subject's blood using techniques known to those of ordinary skill in the art. As an example, a blood sample may be centrifuged at 700 rpm for 20 minutes and the platelet-rich plasma upper layer removed. Platelet density may be determined using a cell count as known to those of ordinary skill in the art. The platelet rich plasma may be mixed with collagen and used as a scaffold. The platelet rich plasma may be mixed with any one or more of the scaffold materials of the invention.

In one embodiment, the gel is a hydrogel. A hydrogel is a substance that is formed when an organic polymer (natural or synthetic) is crosslinked via covalent, ionic, or hydrogen bonds to create a three-dimensional open-lattice structure which entraps water molecules to form a gel. A polymer may be crosslinked to form a hydrogel either before or after implantation into a subject. For instance, a hydrogel may be formed in situ, for example, at a repair site. In certain embodiments, a polymer forms a hydrogel within the repair site upon contact with a crosslinking agent. Naturally occurring and synthetic hydrogel forming polymers, polymer mixtures and copolymers may be utilized as hydrogel precursors. See for example, U.S. Pat. No. 5,709,854. In certain embodiments, a hydrogel is a gel and begins setting immediately upon mixture and takes approximately 5 minutes to sufficiently set before closure of the defect and surgery area. Setting time may vary depending on the mixture of gel used and environmental factors.

For instance, certain polymers that can form ionic hydrogels which are malleable may be used to form the hydrogel. For example, a hydrogel can be produced by cross-linking the anionic salt of alginic acid, a carbohydrate polymer isolated from seaweed, with calcium cations, whose strength increases with either increasing concentrations of calcium ions or alginate. Modified alginate derivatives, for example, which have an improved ability to form hydrogels or which are derivatized with hydrophobic, water-labile chains, e.g., oligomers of €-caprolactone, may be synthesized. Additionally, polysaccharides which gel by exposure to monovalent cations, including bacterial polysaccharides, such as gellan gum, and plant polysaccharides, such as carrageenans, may be crosslinked to form a hydrogel. Additional examples of materials which can be used to form a hydrogel include polyphosphazines and polyacrylates, which are crosslinked ionically, or block copolymers such as PLURONICS™ (polyoxyalkylene ether) or TETRONICS™ (nonionic polymerized alkylene oxide), polyethylene oxide-polypropylene glycol block copolymers which are crosslinked by temperature or pH, respectively. Other materials include proteins such as fibrin, polymers such as polyvinylpyrrolidone, hyaluronic acid and collagen. Polymers such as polysaccharides that are very viscous liquids or are thixotropic and form a gel over time by the slow evolution of structure, are also useful.

In another embodiment, the gel is hyaluronic acid. Hyaluronic acid, which forms an injectable gel with a consistency like a hair gel, may be utilized. Modified hyaluronic acid derivatives are particularly useful. Hyaluronic acid is a linear polysaccharide. Many of its biological effects are a consequence of its ability to bind water, in that up to 500 ml of water may associate with 1 gram of hyaluronic acid. Esterification of hyaluronic acid with uncharged organic moieties reduces the aqueous solubility. Complete esterification with organic alcohols such as benzyl renders the hyaluronic acid derivatives virtually insoluble in water, these compounds then being soluble only in certain aprotic solvents. When films of hyaluronic acid are made, the films essentially are gels which hydrate and expand in the presence of water.

As used herein, the term “pharmaceutically acceptable” means a non-toxic material that does not interfere with the effectiveness of the biological activity of the scaffold material or repair material. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term “carrier” denotes an organic or inorganic ingredient, natural or synthetic, with which the scaffold material is combined to facilitate the application. The components of the pharmaceutical compositions also are capable of being co-mingled with the device of the present invention, and with each other, in a manner such that there is no interaction which would substantially impair the desired pharmaceutical efficacy.

Now referring to FIGS. 6A-6C, the present disclosure includes an example of a surgical procedure which may be performed using the systems and methods disclosed. Prior to insertion of the scaffold 14, the affected extremity is prepared and draped in the standard sterile fashion. A tourniquet may be used if indicated. In FIG. 6A, after diagnostic arthroscopy is performed, the ruptured ligament 2 is identified and defined, the tissue ends 7, 9 are pretreated, either mechanically or chemically. The suture 12 is connected to the fixation device 8.

In FIG. 6B, prior to entering the repair site, the arthroscopic equipment 30 attaches the suture 12 to the scaffold 14. The scaffold 14 may be treated with a repair material. In one embodiment, the scaffold 14 may also be pre-treated in antibiotic solution prior to implantation. The arthroscopic equipment 30 is configured to contain the scaffold 14 and the suture 12. During implantation, the arthroscopic equipment 30 introduces the scaffold 14 and the suture 12 into the tissue defect. In the illustrated embodiment, the arthroscopic equipment 30 introduces the repair device by pushing or releasing the repair device from the container into the repair site.

In the illustrated embodiment, the suture 12 is then connected to the ruptured end of the ligament 2 at the first end 16. In one embodiment, the suture 12 is placed through the ruptured end of the ligament 2 using a whip-stitch. In FIG. 6B, the fixation device 8 is passed through a bone, carrying suture 12. The fixation device 8 and the suture 12 is attached to the bone.

In FIG. 6C, the arthroscopic equipment 30 positions the scaffold 14 along the suture between the ruptured ends of the ligament 2. In alternative embodiments, the arthroscopic equipment 30 positions the scaffold 14 directly or indirectly onto the femur 4 and/or the tibia 6. The present disclosure may be used by insertion through an open incision. The scaffold 14 is compressible to allow introduction through arthroscopic portals, incisions and equipment.

The scaffold 14 is then bonded to the surrounding tissue using the methods described herein. This can be done by the addition of a chemical agent or a physical agent such ultraviolet light, a laser, or heat. The scaffold 14 may be reinforced by placement of additional sutures or clips. The arthroscopic portals is closed, and a sterile dressing placed. The post-operative rehabilitation is dependent on the type and size of lesion treated, and the tissue involved.

In the present disclosure, a subject includes, but is not limited to, any mammal, such as human, non-human primate, mouse, rat, dog, cat, horse or cow. In certain embodiments, a subject is a human. The present disclosure may also include kits for repair of ruptured or torn ligaments. A kit may include a scaffold of the invention having at least one fixation device attached to the scaffold and instructions for use. The scaffold may further include one or more sutures that attach an fixation device to the scaffold. A kit may further include a container that contains a repair material as described herein.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize or be able to ascertain using no more than routine experimentation, many equivalents to the specific embodiments of the invention described herein. Such equivalents are intended to be encompassed by the following claims.

All references disclosed herein are incorporated by reference in their entirety.

What is claimed:

1. An arthroscopic repair system, comprising:
a tissue healing device configured to repair a ligament at a repair site, the tissue healing device having:
an implantable material configured to be positioned between a ruptured end of the ligament and a bone,
at least one fixation device configured to be secured to the bone, and
at least one suture configured to be threaded through or along the implantable material to position the implantable material between to the ruptured end of the ligament and the bone, the at least one suture being attached to the at least one fixation device; and
arthroscopic equipment sized and shaped to contain the at least one suture and the implantable material, the arthroscopic equipment configured to a) insert the suture through the implantable material, and b) position the implantable material between the ruptured end of the ligament and the bone.

2. The system of claim 1, wherein the implantable material is a scaffold.

3. The system of claim 1, wherein the ligament is an ACL and wherein the implantable material allows cell ingrowth.

4. The system of claim 1, wherein the at least one fixation device is conical in shape.

5. The system of claim 1, wherein the at least one fixation device includes a first end and a second end opposite the first end of the fixation device.

6. The system of claim 5, wherein the at least one fixation device includes an eyelet at the first end of the fixation device.

7. The system of claim 6, wherein the at least one suture is attached to the at least one fixation device through the eyelet.

8. The system of claim 5, wherein the at least one fixation device includes a sharpened tip at the second end of the fixation device.

9. The system of claim 8, wherein the sharpened tip is threaded.

10. The system of claim 1, wherein the at least one fixation device is selected from the group consisting of a screw, a barb, a helical fixation device, a staple, a clip, a snap, and a rivet.

11. The system of claim 1, where the implantable material further comprises a repair material.

12. The system of claim 11, where the repair material is a platelet or plasma.

13. The system of claim 11, wherein the tissue healing device further includes one or more growth factors configured to be disposed within or on the implantable material.

14. The system of claim 11, wherein the implantable material consists essentially of a porous collagen sponge.

15. A system for repair of an anterior cruciate ligament comprising:
an fixation device capable of forming a stable attachment to a first bone at a repair site;
a suture having a first end and a second end, the second end being attachable to a ruptured end of the ligament at the repair site, wherein the ligament is configured to be connected to a second bone;
a scaffold, wherein the scaffold consists essentially of a porous sponge scaffold, wherein the scaffold is threaded onto the suture; and
an elongated delivery member having a channel that extends from a proximal end to a distal end, the suture and scaffold contained within the channel such that the scaffold is positionable along the suture.

16. The system of claim 15, wherein the implantable material allows cell ingrowth.

17. The system of claim 15, wherein the fixation device is conical in shape.

18. The system of claim 15, wherein the fixation device includes a first end and a second end opposite the first end of the fixation device.

19. The system of claim 18, wherein the fixation device includes an eyelet at the first end of the fixation device.

20. The system of claim 19, wherein the suture is attached to the fixation device through the eyelet.

21. The system of claim 18, wherein the fixation device includes a sharpened tip at the second end of the fixation device.

22. The system of claim 21, wherein the sharpened tip is threaded.

23. The system of claim 15, wherein the fixation device is selected from the group consisting of a screw, a barb, an anchor, a helical anchor, a staple, a clip, a snap, and a rivet.

24. The system of claim 15, where the scaffold further comprises a repair material.

25. The system of claim 24, where the repair material is a platelet or plasma.

26. The system of claim 24, further comprising one or more growth factors configured to be disposed within or on the scaffold.

27. The system of claim 24, wherein the scaffold consists essentially of a porous collagen sponge.

28. The system of claim 15, further comprising a plunger configured to push the suture and the scaffold into the repair site.

29. The system of claim 15, further comprising a guiding suture that extends out of the distal end of the elongated delivery member, the guiding suture configured to pull and position the suture and the scaffold into the repair site.
